(19) United States
(12) Patent Application Publication
(10) Pub. No.: US 2008/0003309 A1
(43) Pub. Date: Jan. 3, 2008

(54) Yoghurt Formulation Cosmetic Composition

(76) Inventors: Hye Won Lee, Yongin-si (KR); Chang Hoon Park, Seoul (KR); Sang Hoon Han, Suwon-si (KR); Gae Won Nam, Yongin-si (KR)

Correspondence Address: SUGHRUE MION, PLLC, 2100 PENNSYLVANIA AVENUE, N.W., SUITE 800, WASHINGTON, DC, 20037, US

(21) Appl. No.: 10/598,692

(22) PCT Filed: Sep. 6, 2004
(86) PCT No.: PCT/KR04/02253

§ 371 Date: May 15, 2007

(30) Foreign Application Priority Data
Mar. 11, 2004 (KR) 10-2004-0016589

(51) Int. Cl. A61K 36/00; A61K 131/00; A61K 36/23; A61K 36/73; A61K 36/736; A61K 36/81; A61K 47/48; A61Q 19/00

(52) U.S. Cl. 424/735; 424/725; 424/765; 424/777; 514/782

(57) ABSTRACT

The present invention relates to a cosmetic composition with appearance and texture of curd yoghurt realized by containing xanthan gum and mannan. In addition, the present invention can provide skin moisturizing and softening effects improved by further containing vegetable or fruit, or vitamins or yoghurt powder.

### BACKGROUND OF THE INVENTION

1. Field of the Invention

The present invention relates to a cosmetic composition with curd yoghurt formulation, containing xanthan gum and mannan. More particularly, the present invention relates to a cosmetic composition wherein appearance and texture of curd yoghurt are realized in industry by containing xanthan gum and mannan and which can be formulated into a pack.

2. Description of Prior Art

Yoghurt is a kind of fermented milk and produced by fermenting the sterilized mammal milk with lactic acid bacteria and then by adding spices, sweetening and the like thereto, depending on one's taste, to be suitable for drinking. This yoghurt is generally divided into liquid fermented milk and concentrated fermented milk, which is again divided into “curd yoghurt” which is scooping or squeezing-type and “drinking yoghurt” which is drinking-type.

In recent, yoghurt has been used as a skin-care pack. This pack forms a temporary film on the face, to block air inflow and moisture evaporation. Thus, it can soften the skin and enable active ingredients to penetrate into the skin. Various materials have been and are applied to the pack formulation. Recently, there have been many studies on natural materials for skin-care pack.

In the home, pack-using massages have been widely done. As a material for pack herein, food articles such as fruits and grains have been mainly used. Particularly, yoghurt is on the increase in application to a pack. Yoghurt has soft texture and contains various vitamins and qualitative proteins, to be expected for skin moisturizing effect and skin softening effect. However, on directly applied onto the skin, yoghurt may trickle down and cause irritations to the skin. On this account, yoghurt needs gauze in application as a pack, to cause cumbersome.

Under these circumstances, in order to formulate yoghurt as a cosmetic having the usefulness thereof as it is and to improve usability onto the skin, the present inventors have been extensively studied. For the above purpose, we have made many efforts to provide a yoghurt-type pack that can be easily applied onto the skin. We have investigated various polymers and screened the combination thereof in order to realize the same appearance and texture as those of curd yoghurt.

As a result thereof, we found that combination of xanthan gum and mannan can realize emulsions having the same lumpy texture as that of yoghurt and stability, and thus have completed the present invention. In addition, we confirmed that skin moisturizing and softening effects can be obtained by adding extracts of fruits or vegetables such as strawberry, banana, apple, kiwi fruit, broccoli, berries (blueberry, raspberry, etc.), potato, carrot, melon, pineapple and peach; said fruit or vegetable crushes; or milk, vitamins or yoghurt powder. Also, we confirmed that addition of said extracts can improve fragrance and feeling of the cosmetics.

Therefore, an object of the present invention is to provide a cosmetic composition with similar appearance and texture to those of curd yoghurt.

Further, another object of the present invention is to provide a cosmetic composition with skin moisturizing and softening effects, containing fruit or vegetable extracts, or milk, vitamins or yoghurt powder.

A further object of the present invention is to provide a cosmetic composition with pack formulation, which can be directly applied onto the skin without separate treatment.

### SUMMARY OF THE INVENTION

The cosmetic composition according to the present invention is characterized in that it contains xanthan gum and mannan. That is, the present invention provides a cosmetic composition with curd yoghurt-type pack formulation by containing xanthan gum and mannan.

More particularly, said xanthan gum and mannan may be formulated to an emulsion-type cosmetic, so as to realize appearance and texture of yoghurt in a cosmetic.

Said xanthan gum and mannan may be employed in a ratio of 1:4˜4:1 by weight. Within said range, the obtained texture may be the most similar to that of yoghurt and formulation into a pack can be easily processed.

Additionally, the present invention provides a cosmetic composition with appearance and texture of yoghurt realized by containing xanthan gum and mannan. Particularly, said cosmetic may be formulated into an oil-in-water emulsion. More particularly, it may be formulated into a pack.

Said cosmetic composition according to the present invention may contain one or more selected from fruits and vegetables as an edible vegetable ingredient.

And, said cosmetic composition may contain one or more selected from vitamins, milk and yoghurt powder. Preferably, it may contain both said edible vegetable ingredient and yoghurt powder.

Said fruit and vegetable ingredient may be employed in the form of extracts or juices or crushes.

Said edible fruit and vegetable ingredient may preferably include strawberry, apple, apricot, tomato, banana, kiwi fruit, broccoli, berries (blueberry, raspberry, etc.), potato, carrot, melon, pineapple, peach and the like. Said materials may be employed as it is without any treatment or submitted to a pre-treatment, if necessary. For example, potato may be boiled before use, and fruits may be simmered and employed with or without seeds. One skilled in this art can easily process said vegetable ingredient depending on its feature.

Said edible vegetable ingredient may be employed in the various forms of extracts, juices, crushes and the like. One skilled in this art, in consideration of product's feature, consumer's fancy or using peculiarity, can select this form employed. Further, it may be apparent, to those skilled in this art, to change this form, if necessary.

### DETAILED DESCRIPTION OF THE INVENTION

In this invention, the term “curd yoghurt” refers to a concentrated yoghurt having viscosity and exemplified as scooping yoghurt or squeezing yoghurt.

And, the term “pack formulation” in this invention refers to a formulation for forming a film on the skin, after applied thereon. Thus, preferable pack forms a temporary film on the skin, to block air inflow and moisture evaporation and to enable active ingredients contained therein to penetrate into the skin.

Said pack may include a formulation to be directly applied onto the skin or a formulation produced by coating with or applying said cosmetic composition on gauze or paper or permeable membrane such as non-woven fabric.

Said pack formulation embodied by the present invention has feature of curd yoghurt and improved usability, and thereby good stickiness without trickling-down. That is, it may be convenient for use.

In production for a pack, various polymers may be employed for convenient use. These polymers may increase viscosity of a cosmetic or block destruction of emulsion particles, so as to improve stability of the emulsion. And, these polymers, depending on their properties, may have significant effects on forming texture of the emulsion.

The emulsion of the present invention has lumpy texture, i.e. feeling just as soft fleshes fall into the hands; different from general emulsions having smooth texture of whitening appearance. Nevertheless, the emulsion has good stability.

This formulation was developed by screening polymer combinations in consideration of both texture and stability.

The polymers employed herein were carbomer, polyacrylamide, polyvinyl alcohol, mannan, xanthan gum and starch, which have been widely used in general cosmetics. Polymer combinations were incorporated into emulsions and then synergic effects were compared. The results confirmed that emulsion obtained by using combination of xanthan gum and mannan exhibited similar lumpy texture to that of yoghurt and formed stable formulation.

Further, the present invention improved usability as a pack formulation by controlling combinational ratio of xanthan gum and mannan. Thus, the cosmetic composition of the present invention is characterized in that it may contain said xanthan gum and mannan in an amount of 0.02˜10% by weight and preferably 0.2˜2.0% by weight based on the total weight of composition. If the amount is less than 0.02% by weight, it may be difficult to obtain lumpy texture. While, if the amount is more than 10% by weight, it may be out of viscosity range for cosmetics.

The cosmetic curd yoghurt according to the present invention may have the same or higher viscosity than that of edible curd yoghurt. Said viscosity may be controlled depending on the purpose of cosmetic or skin type. One skilled in this art may formulate a pack with viscosity suitable for the purpose, if necessary.

Addition of starch into the combination of xanthan gum and mannan, as confirmed in the below examinational example, can improve appearance and texture of cosmetics. Starch may be incorporated in a ratio of 0˜10 to 1 weight of the combination of xanthan gum and mannan.

The present invention can provide skin moisturizing and softening effects improved by further containing vegetable extracts or fruit extracts or yoghurt powder. That is to say, addition of extracts of fruits such as strawberry that are abundantly incorporated in edible curd yoghurt; or vegetable extracts; or yoghurt powder can improve skin moisturizing and softening effects. Such an addition can exhibit synergic effect in moisturizing and softening actions and realize furthermore similar fragrance and feeling to those of edible yoghurt.

Vegetables and fruits contain rich vitamins, and particularly strawberry contains rich monosaccharides including glucose and fructose, so as to refresh and animate dried and rough skin. And, yoghurt contains vitamin B2 and qualitative proteins, to be expected to provide a cosmetic effect for elastic and bright skin. Thus, the cosmetic composition according to the present invention is characterized in that it may further contain strawberry extract or yoghurt powder.

### BRIEF DESCRIPTION OF THE DRAWINGS

FIG. 1 shows changes in skin hydration with the lapse of time, after applying the cosmetics of Examples 1˜5 and Comparative Example 1 onto the forearm.

FIG. 2 shows changes in skin hydration with the lapse of time, after applying the cosmetic of Example 3 onto the face (brow, frontal cheek, cheek, chin).

FIG. 3 shows changes in skin hydration with the lapse of time, after applying the cosmetic of Example 4 onto the face (brow, frontal cheek, cheek, chin).

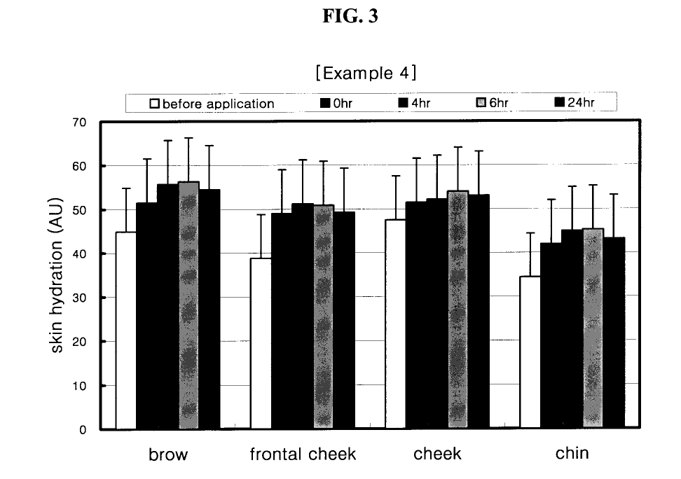

FIG. 4 shows changes in skin hydration with the lapse of time, after applying the cosmetic of Example 5 onto the face (brow, frontal cheek, cheek, chin).

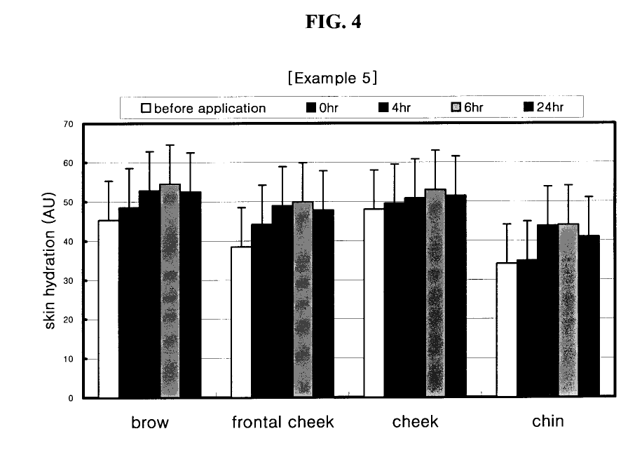

### PREFERRED EMBODIMENT OF THE INVENTION

The present invention will be described in more detail by way of the following examples, which should not be considered to limit the scope of the present invention.

### EXPERIMENTAL EXAMPLE 1

### Searching and Screening of Formulation

Emulsions were produced by combining, as shown in Table 1, polymers such as carbomer, polyacrylamide, polyvinyl alcohol, starch, xanthan gum and mannan which have been widely used in general cosmetics, and estimated for their properties.

While, in the production of the emulsions, decamethylcyclopentasiloxane, hydrogenated polydecene, vegetable squalane, cetyloctanoate, polysorbate 60, sorbitan stearate and sorbitan sesquioleate were added to an oily phase, and distilled water, glycerin and EDTA.2Na were added to an aqueous phase. The emulsions were produced by the conventional method for cosmetics.

For the emulsions produced, experiment was performed in order to confirm lumpy texture thereof.

TABLE 1
Polymer combinations (wt %)

| Combi- | Xanthan | | | Polyacryl- | | Polyvinyl |
|---|---|---|---|---|---|---|
| nation | gum | Mannan | Carbomer | amide | Starch | alcohol |
| #1 | 0.1 | 0.1 | — | — | — | — |
| #2 | 0.1 | — | 0.1 | — | — | — |
| #3 | 0.1 | — | — | 0.1 | — | — |
| #4 | 0.1 | — | — | — | 1.0 | — |
| #5 | 0.1 | — | — | — | — | 1.0 |
| #6 | — | 0.1 | 0.1 | — | — | — |
| #7 | — | 0.1 | — | 0.1 | — | — |
| #8 | — | 0.1 | — | — | 1.0 | — |
| #9 | — | 0.1 | — | — | — | 1.0 |
| #10 | — | — | 0.1 | 0.1 | — | — |
| #11 | — | — | 0.1 | — | 1.0 | — |
| #12 | — | — | 0.1 | — | — | 1.0 |
| #13 | — | — | — | 0.1 | 1.0 | — |
| #14 | — | — | — | 0.1 | — | 1.0 |
| #15 | — | — | — | — | 1.0 | 1.0 |
| #16 | 0.2 | 0.1 | — | — | — | — |
| #17 | 0.1 | 0.2 | — | — | — | — |
| #18 | 0.4 | 0.1 | — | — | — | — |
| #19 | 0.1 | 0.4 | — | — | — | — |
| #20 | 0.1 | 0.1 | 0.1 | — | — | — |
| #21 | 0.1 | 0.1 | — | 0.2 | — | — |
| #22 | 0.1 | 0.1 | — | — | 2.0 | — |
| #23 | 0.1 | 0.1 | — | — | — | 5.0 |

Meanwhile, for the emulsions containing the polymer combinations #1˜#23 of Table 1, lumpy texture thereof was compared and the results are shown in Table 2.

TABLE 2
Results of experiment for polymer combinations

| Combination | Appearance and texture |
|---|---|
| #1 | ⊚ |
| #2 | X |
| #3 | X |
| #4 | X |
| #5 | X |
| #6 | X |
| #7 | X |
| #8 | X |
| #9 | X |
| #10 | X |
| #11 | X |
| #12 | X |
| #13 | X |
| #14 | X |
| #15 | X |
| #16 | ◯ |
| #17 | ◯ |
| #18 | ◯ |
| #19 | ◯ |
| #20 | ◯ |
| #21 | ◯ |
| #22 | ⊚ |
| #23 | X |

(Note, ⊚: lumpy, ◯: slight lumpy, X: No lumpy)

As shown in Table 2, lumpy texture was most observed in the combinations of xanthan gum and mannan.

Further, in the observation of appearance and texture depending on a combinational ratio of xanthan gum and mannan, combination #1 had the most similar appearance and texture to those of curd yoghurt. For all that, most of emulsions containing the combinations of xanthan gum and mannan had lumpy texture.

The combinations #20˜23 further contained other polymer together with xanthan gum and mannan, in order to improve usability to the skin. Among them, the combination #22 was the most satisfactory in appearance and texture.

### EXAMPLES 1˜5 AND COMPARATIVE EXAMPLE 1

Based on the results of Experimental Example 1, the combination of xanthan gum and mannan was added to an oil-in-water emulsion. Added amount of the combination was 0.6% by weight based on the total weight of composition, and weight ratio of xanthan gum:mannan was 1:1 (in Example 1 and Examples 3˜5) and 2:1 (in Example 2). The compositions thereof were shown in Table 3.

In detail, in order to confirm effect of the combination of xanthan gum and mannan in the appearance and texture, said combination was not added to the emulsion of Comparative Example 1. Further, in order to confirm skin-moisturizing effect of the emulsion containing fruit extract together with yoghurt powder, Example 3, Example 4 and Example 5 containing strawberry extract, banana extract and carrot extract, respectively were produced and the skin-moisturizing effect thereof was compared (See Table 3). In the examples, said strawberry, banana and carrot extracts (Bioland ltd. Korea) and yoghurt powder (Cosmetochem, Switzerland) were on the market.

Said extracts and powder are not limited in the kinds and the processing by the present examples and can be directly produced in consideration of the purpose and the usage thereof by those skilled in this art. For example, yoghurt powder can be produced by sterilizing low-fat yoghurt or other kind of yoghurt depending on skin type and then by drying and pulverizing. And, fruits or vegetables can be treated fitting to formulation.

The following Table 3 relates to oil-in-water (O/W) emulsions and unit is % by weight (wt %).

Examples 1˜5 and Comparative Example 1 were produced by the following method.

### Preparation Method

(1) Oily phase was heated to 70˜75° C. and homogenized.
(2) Aqueous phase 1 was heated to 70˜75° C. to be dissolved and homogenized.

(3) Said (1) was added to said (2) under stirring and at the temperature of 70˜75° C. and sequentially aqueous phase 2 was added and then cooled to 28˜30° C.

TABLE 3

| | Materials | Ex. 1 | Ex. 2 | Ex. 3 | Ex. 4 | Ex. 5 | Comp. Ex. 1 |
|---|---|---|---|---|---|---|---|
| Oily | Cetyloctanoate | 4.0 | 4.0 | 4.0 | 4.0 | 4.0 | 4.0 |
| phase | Hydrogenated polydecene | 4.0 | 4.0 | 4.0 | 4.0 | 4.0 | 4.0 |
| | Vegetable squalane | 3.0 | 3.0 | 3.0 | 3.0 | 3.0 | 3.0 |
| | Polysorbate 60 | 1.20 | 1.20 | 1.20 | 1.20 | 1.20 | 1.20 |
| | Sorbitan stearate | 0.30 | 0.30 | 0.30 | 0.30 | 0.30 | 0.30 |
| | Sorbitan sesquioleate | 0.50 | 0.50 | 0.50 | 0.50 | 0.50 | 0.50 |
| | Preservative | 0.2 | 0.2 | 0.2 | 0.2 | 0.2 | 0.2 |
| | Perfume | 0.05 | 0.05 | 0.05 | 0.05 | 0.05 | 0.05 |
| Aqueous | Distilled water | to 100 | to 100 | to 100 | to 100 | to 100 | to 100 |
| phase 1 | Glycerin | 4.0 | 4.0 | 4.0 | 4.0 | 4.0 | 4.0 |
| | Butylene glycol | 6.0 | 6.0 | 6.0 | 6.0 | 6.0 | 6.0 |
| | EDTA•2Na | 0.02 | 0.02 | 0.02 | 0.02 | 0.02 | 0.02 |
| | Sucrose | 0.4 | 0.4 | 0.4 | 0.4 | 0.4 | 0.4 |
| | Xanthan gum | 0.3 | 0.4 | 0.3 | 0.3 | 0.3 | — |
| | Mannan | 0.3 | 0.2 | 0.3 | 0.3 | 0.3 | — |
| | Yoghurt powder | — | — | 0.05 | 0.05 | 0.05 | — |
| | Strawberry extract | — | — | 20.0 | — | — | — |
| | Banana extract | — | — | — | 20.0 | — | — |
| | Carrot extract | — | — | — | — | 20.0 | — |
| | Preservative | 0.2 | 0.2 | 0.2 | 0.2 | 0.2 | 0.2 |
| Aqueous | Starch | — | — | 2.5 | 2.0 | 1.0 | 2.5 |
| phase 2 | | | | | | | |

Appearance and texture of Examples 1˜5 and Comparative Example 1 are shown in Table 4. Among them, Examples 1 and 3˜5 had the most similar appearance and texture to those of curd yoghurt.

TABLE 4

| Test samples | Appearance and texture |
|---|---|
| Example 1 | ⊚ |
| Example 2 | ◯ |
| Example 3 | ⊚ |
| Example 4 | ⊚ |
| Example 5 | ⊚ |
| Comparative Example 1 | X |

(Note, ⊚: lumpy, ◯: slight lumpy, X: No lumpy)

### EXPERIMENTAL EXAMPLE 2

### Evaluation for Skin Hydration

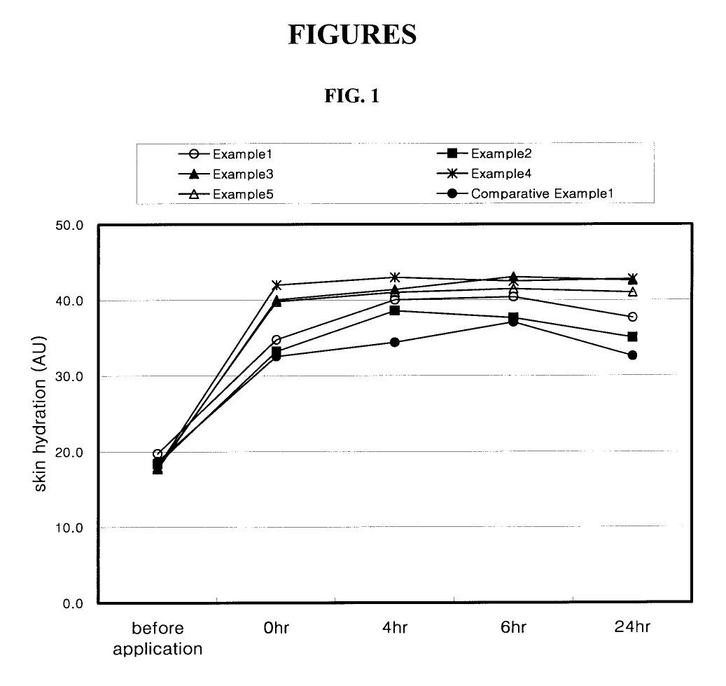

Skin hydration was evaluated by each fifteen (15) of females and males complaining or having dry skin. They stayed in an incubator of 24˜26° C. and relative humidity 40˜50%, for one hour. Then, on their forearms, the cosmetics of Examples 1˜5 and Comparative Example 1 were applied and skin hydration was compared. Before and 0, 4, 6 or 24 hours later after application, skin hydration was measured by Corneometer (Courage & Khazaka, Germany). The results are shown in FIG. 1.

In a separate way, the cosmetics of Examples 3˜5 were applied onto the faces of the subjects, i.e. brows, front cheeks, cheeks and chins. Before and 0, 4, 6 or 24 hours later after application, skin hydration was measured by Corneometer. Herein, the term “front cheek” refers to the front of cheek, on eyes fronting forward, while “cheek” refers to the side of cheek.

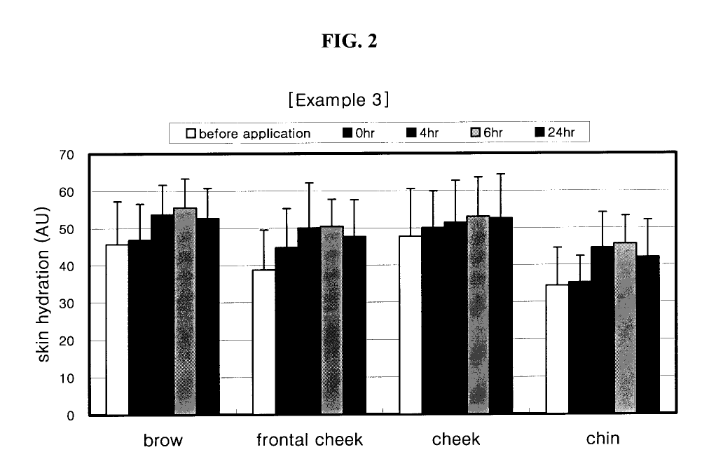

The results are shown in FIGS. 2˜4.

Further, when the test was completed, the subjects were answered to the questions, to perform subjective evaluation, apart from mechanical evaluation. The results are shown in Table 5. The questions were divided to four grades: Very Good; Good; Average; and Unsatisfactory.

TABLE 5
Answer to the questions for skin-moisturizing effect

| | Number of the subjects showing response | | | |
|---|---|---|---|---|
| Test samples | Very good | Good | Average | Unsatisfactory |
| Example 1 | 2 | 7 | 4 | 2 |
| Example 2 | 2 | 7 | 5 | 1 |
| Example 3 | 6 | 5 | 3 | 1 |
| Example 4 | 6 | 7 | 1 | 1 |
| Example 5 | 5 | 6 | 3 | 1 |
| Comparative Example 1 | 1 | 7 | 6 | 1 |

The results of FIG. 1 indicated that the cosmetics of Examples 3˜5 had relatively higher value in skin hydration after application thereof than the cosmetics of Examples 1 and 2. Further, as shown in FIGS. 2˜4, after application of each cosmetic of Examples 3˜5 onto the face, skin hydration increased with the lapse of time. This increase in skin hydration was continuously observed 24 hours later.

These results were confirmed by answering to the questions. As shown in Table 5, the cosmetics of Examples 3˜5 containing fruit and vegetable extracts were relatively excellent in skin moisturizing.

As above described, the present invention relates to a cosmetic composition with appearance and texture of curd yoghurt realized by containing xanthan gum and mannan, which can be formulated into a pack. In addition, the present invention can provide skin hydration increased by further containing fruit or vegetable extracts or yoghurt powder, so as to provide improved skin moisturizing effect.

1. A cosmetic composition with curd yoghurt formulation, characterized in that it contains xanthan gum and mannan.

2. The cosmetic composition according to claim 1, wherein said xanthan gum and mannan are contained in a ratio of 1:4˜4:1 by weight.

3. The cosmetic composition according to claim 1, wherein said xanthan gum and mannan are contained in an amount of 0.02˜10% by weight based on the total weight of composition.

4. The cosmetic composition according to claim 1, which further contains starch in a ratio of 0˜10 to 1 weight of combination of xanthan gum and mannan.

5. The cosmetic composition according to claim 1, which further contains one or more selected from the group consisting of one or more ingredients selected from the group consisting of vegetables and fruits; and one or more ingredients selected from the group consisting of milk, vitamins and yoghurt powder.

6. The cosmetic composition according to claim 5, wherein said vegetable and fruit ingredients are contained in the form of extracts or juices or crushes.

7. The cosmetic composition according to claim 6, wherein said vegetables and fruits are selected from the group consisting of strawberry, apple, apricot, tomato, banana, kiwi fruit, broccoli, berries, potato, carrot, melon, pineapple and peach.

8. The cosmetic composition according to any one of claims 1 to 7, which has a pack formulation.

9. A method for preparing an oil-in-water emulsion-type cosmetic with curd yoghurt formulation, which comprises the step of adding xanthan gum and mannan into an aqueous phase, in a ratio of 1:4˜4:1 by weight and then formulating to an emulsion.
